(19) United States
(12) Patent Application Publication
(10) Pub. No.: US 2004/0151765 A1
(43) Pub. Date: Aug. 5, 2004

(54) Methods and compositions for wound management

(76) Inventors: Branson W. Ritchie, Athens, GA (US); Anthony C. Capomacchia, Bishop, GA (US); Richard E. Wooley, Athens, GA (US)

Correspondence Address: WOMBLE CARLYLE SANDRIDGE & RICE, PLLC, P.O. BOX 7037, ATLANTA, GA, 30357-0037, US

(21) Appl. No.: 10/739,841

(22) Filed: Dec. 18, 2003

Related U.S. Application Data
(63) Continuation-in-part of application No. 09/955,657, filed on Sep. 18, 2001.
(60) Provisional application No. 60/435,413, filed on Dec. 19, 2002.

(51) Int. Cl. A61K 31/573; A61K 31/56; A61L 15/00

(52) U.S. Cl. 424/445; 514/179

(57) ABSTRACT

The present invention provides methods for wound management that comprise contacting a wound of a patient with an effective amount of a therapeutic composition comprising a chelating agent, a pH buffering agent, an antimicrobial agent, Vitamin E and a carrier and a surfactant. The antimicrobial agent(s) has increased antimicrobial activity because of the synergy with the chelating agent and maintenance of the treated area at a pH suitable for sustained antibiotic activity so that the antimicrobial agent can be used in effective doses that are less than in the absence of the chelator. The Vitamin E promotes tissue repair, reducing the likelihood of opportunistic infection, improving wound healing and reducing pain sensation. The wound management methods of the present invention further include contacting the wound with a medical dressing having an effective amount of the composition deposited thereon. The present invention further provides methods for use of a therapeutic composition a mouth rinse for repairing a wound of the oral mucosa.

The present invention is a continuation-in-part of non-provisional U.S. application Ser. No. 09/955,657 filed Sep. 18, 2001, and also claims benefit of provisional U.S. application Ser. No. 60/435,413 filed Dec. 19, 2002, both of which are incorporated herein by reference in their entireties.

### FIELD OF THE INVENTION

The present invention relates generally to methods and compositions for managing wounds, including surface wounds or skin lesions of a human or animal patient. The present invention further relates to methods useful in treating wounds infected by microorganisms. The present invention also relates to methods of promoting the repair of wounds of mouth and the skin surfaces.

### BACKGROUND

The outermost layers of the skin form a physical barrier that protects an animal from microbial invasion and the establishment of opportunistic infections. Injuries to the skin, for instance an abrasion, incision, laceration, a burn from thermal, radiant or chemical exposure or a necrotic lesion of the surface tissue, destroy the integrity of the cornified layer, epidermis or dermis and will allow microbes to penetrate into the underlying tissues. An established infection can become systemic and ultimately life-threatening.

A breach in the skin combined with a compromised immune system means that burn patients are highly susceptible to opportunistic infections. Of about seventy thousand bum victims per year serious enough to require hospitalization, as many as ten thousand will die, usually from a nosocomial infection. Many burn survivors will suffer permanent disfigurement from the tissue damage that accompanies microbial infections. Greater treatment needs and prolonged care of bums patients in the hospital also mean that infections represent a significant financial drain. Long-term recuperative costs can also be significant.

Ulcers are exposed surface lesions of the skin or a mucoid layer such as the lining of the mouth, where inflamed and necrotic tissue sloughs off. This exposed tissue is also highly susceptible to opportunistic microbial invasion. In this instance, the primary infected site is localized and best treated by a topical application of an antimicrobial agent, sometimes supplemented with systemic antibiotic administration. Although tissue damage is not usually as great as for a bum, infected ulcers are discomforting to the patient, disfiguring and also life-threatening if leading to a systemic infection.

The repair of damaged skin requires an ongoing process of physiologic debridement, development of highly vascular granulation tissue that provides wound protection, wound closure through contraction and epithelial migration to provide long-term surface protection. The interval between wounding and skin repair depends on the location of the injury, the degree of damage, the apposition of the wound edges, the medical condition of the patient and the type and severity of wound contamination with foreign debris and microbes.

Trauma and necrosis of the skin decreases vascularization to a wound and slows the influx of immunologic proteins and white blood cells. The severity of the damage and number of invading microbes determine whether or not a clinical infection occurs. While clinicians frequently focus on the type of microbes that may contaminate a wound, some studies suggest that the number of invading microbes is more important than the species. Proliferating microbes cause additional and accelerated tissue damage through both direct (toxins and cellular damage) and indirect (edema and accumulation of pus) impairment of vascular supply. These changes further impair access of immune system components to the wound as well as reducing the clearance of necrotic debris and preventing systemically delivered antibiotics from reaching contaminated tissues. Collagenase and proteases that accumulate in association with degenerating inflammatory cells damage connective tissue proteins and further inhibit wound healing.

The wound healing cascade is delayed until the inflammatory and physiologic debridement phases have killed and removed contaminating microbes and necrotic tissues. Experiments in rats demonstrated that infection decreased the bursting strength of a wound (increased likelihood of dehiscence) as well as increasing angiogenesis and vessel thrombosis. While some data suggests infected wounds that heal by second intention are stronger than ones closed primarily, it is generally considered best to resolve and prevent infection.

Macrophages that enter a wound serve a phagocytic function as well as the critical role of stimulating the maturation of fibroblast that are ultimately responsible for skin repair. Epithelial cell proliferation and migration are necessary for re-epithelization. This process is enhanced in a moist environment that is well oxygenated by a viable blood supply.

Initiating treatment in a timely manner is most important to reduce or prevent microbial colonization and additional pathogen-associated wound damage. Contaminating bacteria begin to invade adjacent tissue within 6 hours and most data suggest that a wound should be considered infected when 10⁵ to 10⁷ microbes are present per gram of tissue.

Surgical preparation of the skin surrounding the wound followed by copious flushing under pressure of the wound with solutions that will remove dirt and bacteria while preserving cellular integrity is critical. The two most commonly used wound irrigants are Betadine and NOLVASAN™. Some studies suggest that Betadine solution will reduce the number of bacteria in a wound while others suggest no effect on microbial colonization In vivo data suggests that significant numbers of bacteria can survive in all noncytotoxic concentrations of Betadine solution and Nolvasan. However, other studies have demonstrated that either of these antimicrobials facilitated improved wound healing compared to saline-treated controls. In a human study, flooding a wound with penicillin significantly reduced the incidence of wound infection. Solutions containing ampicillin, neomycin, kanamycin and gentamicin have also been used for flushing wounds. However,, persistent use of antimicrobials or antiseptics can result in the proliferation of multi-drug resistant bacteria within a hospital, an animal facility or among the animal population cared for by a veterinary facility.

There is still a need, however, not addressed in the prior art, for methods of treating and infection susceptible wounds of humans and animals that also promote wound healing. Such methods should also offer relief from pain and provide protection from infection. Safe and effective means of topically administering such compositions to a wound are also desirable. Suitable composition preferably would also have enhanced activity against drug resistant strains of infectious microbes.

### SUMMARY OF THE INVENTION

The present invention addresses the need to manage a wound, such as a burn, a lesion, an incision or a laceration of the skin, or a lesion of the oral mucosa, of an animal or human patient to promote wound repair and reduce the likelihood of infection. Briefly described, the present invention provides methods and compositions for use in the methods for wound management that comprise contacting a wound of a patient with an effective amount of a therapeutic composition comprising a pharmaceutically acceptable chelating agent, a pharmaceutically acceptable pH buffering agent, an antimicrobial agent, Vitamin E a surfactant and a pharmaceutically acceptable carrier. The antimicrobial agent(s) has increased antimicrobial activity because of the synergy with the chelating agent and maintenance of the treated area at a pH suitable for sustained antibiotic activity. The antimicrobial agent can, therefore, be used in effective doses that are less than would be required for the same level of antimicrobial activity in the absence of the chelator. The compositions of the present invention are, therefore, useful in counteracting or preventing an infection or will be more effective against infections caused by drug-resistant strains of microbes.

The Vitamin E promotes tissue repair, thereby reducing the likelihood of opportunistic infection, improving wound healing and reducing pain sensation. The wound management methods of the present invention may further include delivering an effective amount of the therapeutic composition by contacting the wound with a medical dressing having an effective amount of the composition deposited thereon.

The present invention further provides methods suitable for delivering the therapeutic composition, comprising an antimicrobial agent, a chelating agent and a buffer and a surfactant to an oral wound.

Additional objects, features, and advantages of the invention will become more apparent upon review of the detailed description set forth below when taken in conjunction with the accompanying drawing figures, which are briefly described as follows.

### BRIEF DESCRIPTION OF THE FIGURES

FIG. 1. IsoboloGram, illustrating the combined effect of EDTA and neomycin (in 50 mM Tris) on Staphylococcus aureus.

FIG. 2. IsoboloGram, illustrating the combined effect of EDTA and neomycin (in 50 mM Tris) on Pseudomonas aeruginosa.

FIG. 3. IsoboloGram, illustrating the combined effect of EDTA and neomycin (in 50 mM Tris) on Enterococcus faecalis.

FIG. 4. Growth of Staphylococcus aureus on Mueller Hinton agar when treated alone or with combinations of EDTA, water, and neomycin.

FIG. 5. Growth of Pseudomonas aeruginosa on Mueller Hinton agar when treated alone or with combinations of EDTA, water, and neomycin.

FIG. 6. Growth of Enterococcus faecalis on M Enterococcus agar when treated alone or with combinations of EDTA, water, and neomycin.

### DETAILED DESCRIPTION OF THE INVENTION

A full and enabling disclosure of the present invention, including the best mode known to the inventor of carrying out the invention is set forth more particularly in the remainder of the specification, including reference to the Examples. This description is made for the purpose of illustrating the general principles of the invention and should not be taken in the limiting sense.

The present invention addresses the need to manage a wound, such as a burn, a lesion, an incision or a laceration of the skin, or a lesion of the oral mucosa, of an animal or human patient to promote wound repair and reduce the likelihood of infection. The present invention provides methods and compositions for wound management, the methods comprising contacting a wound of a patient with an effective amount of a therapeutic composition comprising a pharmaceutically acceptable chelating agent, a pharmaceutically acceptable pH buffering agent, an antimicrobial agent, Vitamin E, a surfactant and a pharmaceutically acceptable carrier. The antimicrobial agent(s) has increased antimicrobial activity because of the synergy with the chelating agent and maintenance of the treated area at a pH suitable for sustained antibiotic activity. The antimicrobial agent can, therefore, be used in effective doses that are less than would be required for the same level of antimicrobial activity in the absence of the chelator. The compositions of the present invention are, therefore, useful in counteracting or preventing an infection or will be more effective against infections caused by drug-resistant strains of microbes.

The Vitamin E promotes tissue repair, thereby reducing the likelihood of opportunistic infection, improving wound healing and reducing pain sensation. The wound management methods of the present invention may further include delivering an effective amount of the therapeutic composition by contacting the wound with a medical dressing having an effective amount of the composition deposited thereon.

The present invention further provides methods for delivery of a therapeutic composition, comprising an antimicrobial agent, a chelating agent and a buffer and a surfactant for repairing a wound and inhibiting a microbial colonization of the oral mucosa.

Definitions

The term “therapeutically effective” amount of a composition of the present invention is an amount that results in wound repair and/or a reduction in pain sensation at the site of a treated wound and the inhibition or prevention of microbial invasion of the treated wound. A skilled artisan or scientist using routine protocols, such as those disclosed in the Examples below or in the literature, may readily confirm the utility of the compositions described herein.

The term “wound” as used herein refers to a lesion or open wound that can expose underlying epidermal, dermal, muscular or adipoidal tissue to the air. Wounds include, but are not limited to, a puncture wound, an incision, a laceration, a penetrating wound, a perforating wound, a tunnel wound and the like. Wounds also include open wounds that have been sutured or otherwise mechanically closed but have not healed or repaired the break in the skin or oral mucosal layer or of the surface layers of the eye including the conjunctiva and cornea.

The terms “lesion” and “surface lesion” as used herein refer to a circumscribed area of pathologically altered tissue, an injury or wound. Primary lesions are the immediate result of the pathological condition and include, but are not limited to, cuts, abrasions, vesicles, blebs, bullae chancres, pustules, tubercles or any other such condition of the skin or a surface of the mouth, nose, anus or any other orifice of the body of a human or animal, or to the surface layers of the eye including the conjunctiva and cornea., or secondary lesions that later develop from a primary lesion and includes, but is not limited to, fissures and ulcers and other wounds.

The term “wound management” refers to therapeutic methods that induce and/or promote repair of a wound including, but not limited to, arresting tissue damage such as necrotization, promoting tissue growth and repair, reduction or elimination of an established microbial infection of the wound and prevention of new or additional microbial infection or colonization. The term may further include reducing or eliminating the sensation of pain attributable to a wound.

The terms “wound healing” and “wound repair” refer to a process involving tissue growth that partially or totally closes a wound, repairs a breach in the dermis or epidermis and partially or totally restores the barrier properties of the skin or the repair of the surface layers of the eye including the conjunctiva and cornea.

The term “microbial infection” as used herein refers to any pathological presence of at least one bacterial species on or in an injury or lesion to the skin of a human or animal. It is further understood that a “microbial infection” may include any systemic infection that is amenable to inhibition by application of the antimicrobial compositions of the present invention.

The term “burn” as used herein refers to tissue injury of the skin caused by thermal, chemical, or radiation exposure or abrasive friction. A bum may be a “first-degree burn” with superficial damage to the outer cornified layer, a “second-degree burn” with damage extends down into the epidermal layer of cells but is not of sufficient extent that regeneration of the skin is prevented, or a “third-degree burn” where the injury extends below the dermis to the underlying tissue and wherein repair of the skin is not possible without grafting.

The term “ulcer” as used herein refers to an open sore or lesion of the skin or a mucous membrane that involves the sloughing off of inflamed and necrotized tissue and includes, but is not limited to, callous ulcers, chronic leg ulcers, decubitus, denture ulcers of the oral mucosa, traumatic ulcers of the mouth, infections stomatitis of the mouth and any type of secondary lesion that is a breach of the cornified and the epidermal layer of the skin or the mucosal surface of the mouth.

The term “antimicrobial agent” as used herein refers to the compounds and combinations thereof, including bacteristatic or bactericidal compositions or agents, that may be administered to an animal or human and which inhibit the proliferation of a microbial infection.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “chelating agent” as used herein refers to any organic or inorganic compound that will bind to a metal ion having a valence greater than one.

The term “pH buffering agent” as used herein refers to any pharmaceutically acceptable organic or inorganic compound or combination of compounds that will maintain the pH of an antibiotic-containing solution within 0.5 pH units of a selected pH value.

The term “carrier” as used herein refers to any pharmaceutically acceptable solvent of antibiotics, chelating agents and pH buffering agents that will allow a therapeutic composition to be administered directly to a wound of the skin or to the oral mucosa. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal. The term “carrier” is understood not to include surfactants such as detergents, non-ionic surfactants such as lecithin, and the like.

The term “surfactant” as used herein refers to any detergent or other pharmaceutically acceptable non-ionic compound that lowers surface tension of an aqueous solution.

One aspect of the present invention provides methods for wound management wherein a wound of a human or animal patient is contacted with an effective amount a therapeutic composition comprising a chelating agent, a buffer, an antimicrobial agent, Vitamin E, a surfactant and a carrier. More than one antimicrobial agent may be used to inhibit the proliferation of a single invasive organism, or a mixed population of invasive organisms. The antimicrobial agent(s) should be selected after determining the composition and antibiotic resistance spectrum of the invading microbial population.

Administration of the compositions of the present invention to a wound results in accelerated wound repair with reduced or no sepsis, as described in Examples 5-14 below. Even with wounds that penetrated the dermal layer, there is reduced pain sensation, more extensive and quicker tissue growth and less overall discomfort to the patient. An additional benefit is that the tissue repair restrict opportunistic infections that would otherwise prolong the period of wound healing, increase the extent of the wound or even develop to threaten the life of the infected patient.

Before applying the therapeutic composition to the patient, the wound can be debrided to clean the wound of necrotic or infected tissue. Debridation may be mechanical by cutting or pulling away damaged tissue from the wound or, if readily inaccessible, other methods including, but not limited to, the application of sterile maggots may be used. Optionally, the wound may be prewashed before the application of the therapeutic composition using a composition comprising a chelating agent having a concentration from about 1 mM to about 250 mM, a buffering agent having a concentration of about 10 mM to about 250 mM and a detergent having a concentration from about 1 to about 30% v/v as given, for instance, in Example 15 below. In one embodiment, the composition comprises about 8 mM EDTA and 20 mM Tris with between about 2% and about 30% v/v of cocamidopropyl betaine

The therapeutic compositions used in the methods of wound management herein described may be applied to a wound by any number of methods including as a lavage where the wound is washed or irrigated. In one embodiment, for example, the compositions are absorbed onto the surface of the fibers of a wound dressing before or during the treatment, ensuring that while the wound is ventilated it is still subject to contact with the therapeutic compositions for a prolonged period.

In various embodiments of the methods of the present invention, the treated wound is in an oral mucosal surface of the patient. In this instance, the therapeutic composition can be applied as a mouth wash or rinse or in combination with a dressing that may be secured over the wound. In other embodiments, the therapeutic compositions of the invention are ophthalmic compositions suitable for administering to the surface of an eye for the repair or healing of a wound to the conjunctiva or corneal surface. The therapeutic compositions of the present invention may also be used as a bath for the total or partial immersion of a human or animal for the treatment of multiple skin lesions such as for managing or burnt foot, or hand, or large wound, of a human or animal.

The pharmaceutically acceptable chelating agent of the therapeutic compositions of the present methods may be selected from ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate or any other pharmaceutically acceptable chelating agent, salt or combination thereof, known to one of ordinary skill in the art, and which will chelate divalent metal ions such as, but not only, Ca²⁺, Mg²⁺, Mn²⁺, Fe²⁺, and Zn²⁺. The chelating agent, when delivered to a wound of a human or animal patient will have a concentration between from about 1 mM to about 250 mM, more preferably from about 1 mM to about 100 mM, most preferably from about 1 mM to about 50 mM. In a preferred embodiment the chelating agent is EDTA having a concentration of about 8 mM.

The therapeutic compositions of the present invention also include a pharmaceutically acceptable pH buffering agent that preferably will maintain the pH of the antimicrobial composition, when delivered to the skin injury or skin lesion, to between about pH 7.0 and about pH 9.0. A pH buffering agent may be selected from, but is not limited to, Tris (hydroxymethyl) aminomethane (tromethaprim; TRIZMA base), or salts thereof, phosphates or any other buffering agent such as, for example, phosphate-buffered saline that is biologically acceptable. In a preferred embodiment, the pH of the antimicrobial composition in solution is about 8.0. The buffering agent, when delivered to a wound, has an effective dose of between about 5 mM and about 250 mM, more preferably between about 5 mM and about 100 mM, most preferably between about 10 mM and about 100 mM. In a preferred embodiment the buffer agent has a concentration of about 20 mM.

The compositions of the present invention may also comprise at least one antimicrobial agent. The infections that may be treated by the methods and compositions of the present invention may be any opportunistic infection of a wound by a bacterium, or a multiple infection of more than one species of bacteria. Microbial species that may cause infections inhibited by the methods of the present invention include fungi and bacterial species that may cause infections of a burn, lesion, oral mucosal lesion or other wound of a human or animal including, but are not limited to, Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic E. coli, Enterotoxin-producing E. coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P. vulgaris, Pseudomonas aeruginosa, Rhodococcus equi, Salmonella enteridis, S. typhimurium, S. typhosa, Shigella sonnei, S. dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The action of the antimicrobial agent can be either bacteriostatic wherein the antibiotic arrests the proliferation of, but does not necessarily kill, the microorganism or the activity of the antibiotic can be bacteriocidal and kill the organism or a combination of activities. Antibiotics suitable for use in the wound management methods of the present invention include, but are not limited to, β-lactams (penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocins, sulfonamides and trimethoprim, rifampicins, metronidazoles, quinolones, novobiocins, polymixins and Gramicidins and the like and any salts or variants thereof. It also understood that it is within the scope of the present invention that the tetracyclines include, but are not limited to, immunocycline, chlortetracycline, oxytetracycline, demeclocycline, methacycline, doxycycline and minocycline and the like. It is also further understood that it is within the scope of the present invention that aminoglycoside antibiotics include, but are not limited to, gentamicin, amikacin and neomycin and the like.

Techniques to identify the infecting microorganism and to determine the concentration of the antibiotic that will inhibit or kill fifty percent (MIC₅₀) of the organisms will be considered well known to one of ordinary skill in the art and will not require undue experimentation. The techniques to determine the antibiotic sensitivity of a bacterial isolate, and the methods of determining the synergistic effect of adding a chelating agent to a solution of an antibiotic are described in Manual of Methods for General Microbiology, Eds: Gerhardt et al., American Society of Microbiology, 1981, and incorporated herein in its entirety by reference.

Before the application to a wound of a composition that includes an antibiotic, it is be useful to identify the species and the antibiotic sensitivity spectrum of the invasive microbe(s). Routine tests well known to one of ordinary skill in the art, including determining the MIC and FIC of antibiotics in the absence and/or presence of a chelating agent may be used and the amount of the antimicrobial composition may be adjusted accordingly so as to inhibit growth of the microorganism. The concentrations and amounts of the antimicrobial agent and chelating agent may be adjusted to levels that are physiologically accepted by the exposed tissue of the injury or lesion and effective against the microbial infection of the skin injury or skin lesion. In one embodiment of the present invention, the concentration of the antibiotic is in the range of about 0.04 mg/ml to about 25 mg/ml and the concentration of the chelating agent in the carrier is in the range of about 0.1 mM to about 100.0 mM.

In various embodiments, the antibiotic is a penicillin, an aminoglycoside, a vancomycin, a chloramphenicol, an erythromycin, a tetracycline, gentamicin, nalidixic acids, or a streptomycin. In another embodiment the antibiotic is tetracycline. In a preferred embodiment of the present invention, the antibiotic is neomycin. In another embodiment of the present invention, the antibiotic is amikacin. In yet another embodiment, the antibiotic is gentamicin. However, a combination of antibiotics may be used depending upon the antibiotic resistance profiles of the microbial population of the wound.

The therapeutic compositions for use in the methods of wound management also comprise a surfactant that can useful in cleaning a wound or contributing to bactericidal activity of the administered compositions. Suitable surfactants include, but are not limited to, phospholipids such as lecithin, including soy lecithin and detergents. Preferably, the surfactant selected for application to a wound or skin surface is mild and not lead to extensive irritation or promote further tissue damage to the patient.

Suitable nonionic surfactants which can be used are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. A preferred surfactant is polypropylene glycol ethoxylates with a preferred concentration of between about 5% wt % and about 25% wt %. A most preferred surfactant is Pluronic F-127 (Poloxamer 407). In other embodiments of the composition, the surfactant comprises lecithin with or without the addition of Pluronic F-127, the Pluronic F-127 being between about 2 and about 20 wt % for increasing the viscosity or gelling of the compositions.

The therapeutic compositions further include Vitamin E that will promote tissue growth and repair and reduce the pain experienced at the site of the skin injury. By promoting tissue repair, not only is discomfort to the patient reduced, but there may be less scar tissue formation and hence less permanent disfiguring of the patient. Furthermore, faster healing of a skin injury or lesion is useful for reducing the likelihood of a nosocomial infection and the problems associated therewith. The Vitamin E, when delivered to a wound of a patient by the methods of the present invention, has a concentration of between about 20 IU/ml and 500 IU/ml, preferably between about 50 IU/ml and 500 IU/ml, more preferably between about 100 IU/ml and about 500 IU/ml. In one embodiment of the therapeutic composition, the Vitamin E has a concentration of about 100 IU/ml. In another embodiment, the concentration is between about 325 IU/ml and about 360 IU/ml.

The therapeutic compositions for use in the methods of the invention preferably include a pharmaceutically acceptable carrier that provides the medium in which are dissolved or suspended the constituents of the compositions. Suitable carriers include any aqueous medium, oil, emulsion, ointment and the like that will allow the therapeutic compositions to be delivered to the target wound without increasing damage to the tissues of the wound.

It is also contemplated that the therapeutic compositions of the invention can be prepared as precursor solutions, or as sterile powders or concentrates that are useful for the extemporaneous preparation of the administered compositions. Optionally, the compositions may further include a preservative to extend the shelf-life of the composition. A particularly useful preservative is scorbic acid, preferably as the sodium or potassium salt. A preferred amount of the preservative is between about 0.1 wt % to about 5 wt %. A more preferred amount is about 0.2 wt %.

Medical dressings suitable for use in the methods of the present invention for contacting a wound with the therapeutic compositions can be any material that is biologically acceptable and suitable for placing over any wound such as a burn, or a surface lesion of the skin or the oral mucosa or teeth of the mouth. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support for the antimicrobial composition of the present invention is KY™ (sodium carboxymethylcellulose 7H 4F (Hercules, Inc., Wilmington, Del.)).

A film, a natural or synthetic polymer, or a rigid or malleable material that is known to one of ordinary skill in the art as being acceptable for insertion in the mouth of a human or animal, and which will place an antimicrobial composition according to the present invention in contact with a tooth or a lesion of the oral mucosa. In one such embodiment of the medical dressing of the present invention, the support is a gauze. The gauze may be absorbent and can be wetted with an antimicrobial composition of the present invention before applying the gauze to an infected wound or other site.

The present invention also contemplates that the gauze may be impregnated with the therapeutic composition and then dried. This allows the impregnated dressing to be stored for later use, or to avoid excessively dampening an injured area. In yet another embodiment of the present invention, a therapeutic composition is absorbed on the surface of the support material of the medical dressing. The composition may be applied to the surface by wetting the surface with a solution of the composition and drying the support to deposit the composition thereon. A concentration of the composition that is effective for promoting wound repair and/or against the proliferation of a microorganism may be attained when the dressing is wetted by the patient's body.

Another aspect of the present invention is therapeutic compositions comprising about 2 to about 50 wt % of a pharmaceutically acceptable chelating agent, about 2 to about 50 wt % of a pharmaceutically acceptable buffering agent, and a surfactant. In one embodiment, the surfactant is a detergent such as, but not limited to, a mild detergent that will avoid irritation to the surface of the skin or other tissue to which the therapeutic composition is applied. For instance, one suitable detergent is cocamidopropyl betaine, but it is contemplated that any detergent known to those of ordinary skill and which will clean a wound or skin surface without triggering an inflammatory reaction or otherwise further extends the extent of injury of the recipient patient can be used. In one embodiment, the detergent is about 3 to about 33 wt % of a therapeutic composition. In another embodiment the surfactant is lecithin.

In various embodiments of the invention, the composition further comprises from about 1 to about 25 wt % of an antimicrobial agent. The embodiments may also include from about 2 to about 50 wt % of Vitamin E and/or from about 2 to about 98 wt % of a pharmaceutically acceptable carrier. Optionally, the therapeutic compositions include a preservative that will increase the shelf-life of the compositions. A typical preservative is scorbic acid, or the salts thereof.

In the compositions of the present invention, the chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate. Preferably, the chelating agent is ethylenediamenetetracetic acid (EDTA).

The various embodiments of the compositions can further comprise 1 to 20 wt % of an anti-inflammatory agent such as, but not limited to dexamethasone.

The antimicrobial agent(s) that may be included in the various embodiments of the compositions include, but are not limited to, a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a gramicidin. More preferably, the antibiotic s selected from the group consisting of a β-lactam, an aminoglycoside, a vancomycin, a chloramphenicol, an erythromycin, a tetracycline, gentamicin, nalidixic acid and a streptomycin. In one embodiment, the antimicrobial agent is oxytetracycline. In another embodiment, the antimicrobial agent is amikacin. In yet another embodiment, the antimicrobial agent is neomycin.

Compositions of the present invention may also include a carrier, as described above, for dissolving or suspending the components of the therapeutic composition.

In various embodiments of the therapeutic compositions, the pharmaceutically acceptable pH buffering agent can be Tris (hydroxymethyl) aminomethane (TRIZMA Base) which, when dissolved in a carrier will have a concentration of between about 5 mM and about 250 mM, preferably between about 5 mM and about 100 mM, more preferably between about 10 mM and about 100 mM. In a most preferred embodiment, the concentration of the buffering agent is about 20 mM.

In various embodiments of the therapeutic compositions, the Vitamin E, when dissolved in a carrier has a concentration of between about 20 IU/ml and 500 IU/ml, preferably between about 50 IU/ml and 500 IU/ml, more preferably yet between about 100 IU/ml and about 500 IU/ml. In one embodiment the concentration of the Vitamin E is between about 325 IU/ml and about 360 IU/ml. Optionally, the Vitamin E may be prepared by dissolving in hydrous lanolin as a carrier in a volume ratio from about 10:1 to about 1:1, preferably having a volume ratio of about 35:15. Preferably, in all of embodiments, the pharmaceutically acceptable carrier is non-allergenic.

Another aspect of the invention is kits that comprise therapeutic compositions as described above, or the components to prepare the compositions, and packaging that includes instructions on how to prepare and use the compositions to manage a wound and promote healing thereof. One embodiment of the invention, therefore, comprises a vessel containing a pharmaceutically acceptable chelating agent, a pharmaceutically acceptable buffering agent suitable for maintaining the pH of the site of a treated wound, a pharmaceutically acceptable antimicrobial agent, Vitamin E, a pharmaceutically acceptable carrier, a surfactant and packaging material. The packaging material comprises instructions directing the use of the kit for preparing the therapeutic composition of the present invention and delivering the composition to a wound or to the mouth of a human or animal to accelerate healing of a wound. The kit may further comprise separate vessels containing Vitamin E and/or surfactant, instructions for adding the Vitamin E to the therapeutic composition and for administering the therapeutic compositions to the animal or human patient.

Even though the invention has been described with a certain degree of particularity, it is evident that many alternatives, modifications, and variations will be apparent to those skilled in the art in light of the present disclosure. Accordingly, it is intended that all such alternatives, modifications, and variations that fall within the spirit and the scope of the invention be embraced by the defined claims.

The following examples are presented to describe preferred embodiments and utilities of the present invention, but should not be construed as limiting thereof.

### EXAMPLE 1

### Determination of Synergistic Actions and Fractional Inhibitory Concentration (FIC) Index

The antibacterial action of combinations of EDTA-Tris and neomycin was measured by a two-dimensional microtiter checkerboard technique described in Gilman et al., The Pharmacological Basis of Therapeutics, eds Goodman and Gilman, 1085-1086 (Macmillan Publishing Co., New York, 1985),. Sabath, L. D, Antimicrob. Agents and Chem. 210-217. (1967) and Sparks et al., Vet. Res. Comm. 18, 241-249 (1994), incorporated herein by reference in their entireties.

Each well of a round-bottomed 96-well microtiter plate was inoculated with 0.05 ml of 2-fold dilutions of neomycin and EDTA in 50 mM Tris. Then 0.05 ml of an 18-hour old culture of a test organism, containing 10⁶ colony-forming units (CFU)/ml, was added to each well. Controls for the culture and media were included in each plate. Plates were covered and incubated at 37° Celsius for 18-24 hours.

Results were plotted as isobolograms for the determination of antagonistic, neutral or additive, or synergistic effects. To generate isobolograms, FICs of the two test solutions were plotted individually on the x-axis and y-axis to determine the effect of combining the two test solutions on bacterial growth. A line that curves away from the zero point and the coordinates indicates antagonism. A straight line indicates neutral or additive effects. Lines that curves toward the zero point and the coordinates are indicative of synergism if there is at least a 4-fold decrease in the MIC of each compound, when used in combination, as compared with the MIC of each test compound alone.

A numerical score or fractional inhibitory concentration (FIC) index was determined. The FIC index is equal to the sum of the values of FIC for the individual drugs:

$$FIC = \frac{MIC of Drug A with Drug B}{MIC of Drug A} + \frac{MIC of Drug B with Drug A}{MIC of Drug B}$$

An FIC index greater than 1.0 indicates an antagonistic interaction, an FIC index of 1.0 indicates addition, and an FIC index of less than or equal to 0.5 indicates synergism between the two test agents.

### EXAMPLE 2

### Inhibition of the Growth of Microorganisms Infecting Burns

The organisms of this study were isolated from human burn patients. They included strains of methicillin resistant Staphylococcus aureus, and vancomycin resistant strains of Pseudomonas aeruginosa and Enterococcus faecalis. The bacterial isolates were propagated in or on Brain Heart Infusion broth (BHI), Mueller-Hinton Broth (MHB), blood agar (BA), Mueller-Hinton agar (MHA), enterococcus agar (EA), or 2× nutrient agar (2×NA).

The EDTA-Tris treatment solutions were prepared from a stock solution containing 0.5 mols/l sodium EDTA and 1.0 mols/l Tris-HC1, pH 8.0. The treatment solutions contained 5mM sodium EDTA and 50 mM Tris-HC1 with or without of neomycin sulfate 1 mg/ml.

Antibiotic resistance profiles were determined by the disc diffusion method on MHA (5). Antibiotics tested included ampicillin (AM-10), chloramphenicol (C-30), ciprofloxacin (CIP-5), kanamycin (K-30), gentamicin (GM-10), nalidixic acid (NA-30), neomycin (N-30), streptomycin (S-10), sulfisoxazole (G-25), tetracycline (Te-30), and vancomycin (Va-30).

Minimal Inhibitory Concentrations (MICs) and Minimal Bactericidal Concentrations (MBCs) for EDTA-Tris and neomycin were determined by the broth-dilution microtiter method in MHB or BHI according to the method of Blair et al., Manual of Clinical Microbiology. p.307 (pub: Am. Soc. Microbiol. Williams and Wilkins, Baltimore 1970), incorporated herein by reference in its entirety.

### EXAMPLE 3

### In Vitro Effect of EDTA-Tris and Neomycin on Enterococcus faecalis, Pseudomonas aeruginosa, and Staphylococcus aureus

2×NA plates were swabbed with 200 ml of an overnight culture containing about 10⁷ colony-forming-units of a test organism. The plates were sampled with multipoint contactors as described in Wooley et al., Am. J. Vet. Res. 35, 807-810 (1974). Each multipoint contactor consisted of an array of 27 mm sewing needles mounted to an aluminum plate measuring 1 mm×50 mm×50 mm. The needles were set 3.5 mm apart. The multipoint contactors were sterilized by autoclaving. To collect samples, a multipoint contactor was touched to an overnight bacterial culture grown on 2×NA as described above. Replicate plates were then inoculated by lightly pressing the needles bearing the test bacteria onto either MHA plates, BA plates or EA plates for Ps. aeruginosa, Staph. aureus and Ent. faecalis respectively. The agar plates were incubated at 37° C. and colonies were counted at 24 hours and 48 hours.

Each strain of microorganism was tested on a control agar plate (plate 1), and on plates wherein the inoculated bacteria were covered with a sterile surgical gauze saturated with 7 ml of: 5 mM EDTA-Tris (plate 2); 5 mM EDTA-Tris and 1 mg/ml neomycin (plate 3); 1 mg/ml neomycin (plate 4); sterile water (plate 5). Samples were taken at 0 mins, and at 30 mins, 1 hour, 2 hours, 4 hours, 6 hours, 8 hours, and 24 hours of incubation.

### EXAMPLE 4

### The Antibiotic Resistance Profiles, MIC and MBC Values For Test Strains of Staph. aureus, Ps. aeruginosa, and Ent. faecalis

The antibiotic resistance profiles and MIC values for test strains of Staph. aureus, Ps. aeruginosa, and Ent. faecalis are shown on Table 1.

TABLE 1
Antibiotic resistance profiles of Staphylococcus aureus,
Pseudomonas aeruginosa, and Enterococcus faecalis.

| | Antimicrobial Agents^{A} | | | | | | | | | | |
|---|---|---|---|---|---|---|---|---|---|---|---|
| | Am | C | Cip | Gm | K | NA | N | S | G | Te | Va |
| Staphylococcus aureus | R^{B} | I | R | S | R | R | R | S | S | S | S |
| Pseudomonas aeruginosa | R | R | I | I | R | R | R | R | R | R | R |
| Enterococcus faecalis | S | R | R | R | R | R | R | R | R | R | R |

^{A}Am = ampicillin;
C = chloramphenicol;
Cip = ciprofloxacin;
K = kanamycin;
Gm = gentamicin;
NA = nalidixic acid;
N = neomycin;
S = streptomycin;
G = sulfisoxazole;
Te = tetracycline;
Va = vancomycin;
^{B}R = resistant;
I = intermediate;
S = sensitive.

Fractional inhibitory concentrations (FICs) and isoboloGrams for the EDTA-Tris-neomycin combination to determine a synergistic, additive, or antagonistic reaction, as described in Example 1, were determined for Staph. aureus, Ps. aeruginosa, and Ent. faecalis. MIC and MBC values for concentrations of neomycin, 20 ampicillin, chloramphenicol, amikacin and oxytetracycline and EDTA administered individually, and the FIC values for Staph. aureus, Ps. aeruginosa, and Ent. faecalis are shown in Table 2 (Columns 2 and 3). MIC values for mixtures of the above antibiotics and EDTA in the presence of each other are shown in Table 2 (Columns 4 and 5 respectively).

TABLE 2
Minimal Inhibitory Concentration (MIC) data
concerning the amounts (mM) of EDTA in 50 mM
Tris and antibiotics (mg/ml) when reacting alone
and in combination against Pseudomonas aeruginosa,
Staphylococcus aureus, and Enterococcus faecalis.

| | MIC | | | | |
|---|---|---|---|---|---|
| | Individually | | | | |
| | Administered | | | | |
| | | EDTA | Co-administered | | FIC |
| | Neomycin | | Neomycin + EDTA | | |
| Ps. aeruginosa | 1.0 | 1.25 | 0.063 | 0.156 | 0.19 |
| Staph. aureus | 3.13 | 1.0 | 1.56 | 0.25 | 0.75 |
| Ent. faecalis | 3.13 | 15.63 | 1.17 | 1.88 | 0.5 |
| | Ampicillin | | Ampicillin + EDTA | | |
| Ps. aeruginosa | 0.49 | 1.25 | 0.123 | 0.156 | 0.38 |
| Staph. aureus | 0.24 | 1.0 | 0.0075 | 0.25 | 0.28 |
| Ent. faecalis | 0.001 | 15.63 | 0.00025 | 7.82 | 0.75 |
| | Chloramphenicol | | Chloramphenicol + EDTA | | |
| Ps. aeruginosa | 12.5 | 1.25 | 1.56 | 0.313 | 0.37 |
| Staph. aureus | 0.39 | 1.0 | 0.39 | 1.0 | 2.0 |
| Ent. faecalis | 0.4 | 15.63 | 0.2 | 3.9 | 0.75 |
| | Amikacin | | Amikacin + EDTA | | |
| Ps. aeruginosa | 0.001 | 1.25 | 0.001 | 1.25 | 2.0 |
| Staph. aureus | 0.12 | 1.0 | 0.03 | 0.5 | 0.75 |
| Ent. faecalis | 2.0 | 15.63 | 1.0 | 7.8 | 1.0 |
| | Oxytetracycline | | Oxytetracycline + EDTA | | |
| Ps. aeruginosa | 0.003 | 1.25 | 0.00075 | 0.313 | 0.5 |
| Staph. aureus | 0.0001 | 1.0 | 0.00005 | 0.5 | 1.0 |
| Ent. faecalis | 0.05 | 15.63 | 0.025 | 3.91 | 0.75 |

*Synergistic reaction (FIC = # 0.5)
Additive reaction (FIC => .05 to # 1.0)
Antagonistic reaction (FIC => 1.0)

The MBC values for EDTA and neomycin were decreased by at least 75% for bacterial killing (MBC) in those situations in which synergistic potentation occurred (Ps. aeruginosa and Ent. faecalis) as shown in Table 3. A decrease of about 50% was observed with Staph. aureus.

TABLE 3
Minimal Bactericidal Concentrations (MBC), of
Staphylococcus aureus, Pseudomonas aeruginosa,
and Enterococcus faecalis reacted with EDTA (mM)
and neomycin (mg/ml) in 50 mM Tris.

| | | Individually | Co- |
|---|---|---|---|
| Bacterial Species | | Administered | administered |
| Staph. aureus | EDTA (mM) | 7.81 | 3.9 |
| | Neomycin (mg/ml) | 3.13 | 1.56 |
| Ps. aeruginosa | EDTA (mM) | 250 | 20.0 |
| | Neomycin (mg/ml) | 5.0 | 0.04 |
| En. faecalis | EDTA (mM) | 250 | 62.5 |
| | Neomycin (mg/ml) | 25.0 | 6.25 |

Specifically in the case of Staph. aureus, the MBC values for EDTA and neomycin when combined were decreased by 50% as compared to the bactericidal effect of each when individually administered.

With Ps. aeruginosa, the MBC values for EDTA and neomycin when in combination were decreased 99.2% compared to when EDTA or neomycin were individually administered. In the case of Ent. faecalis, MBC values of EDTA and neomycin were both reduced 75% compared to when EDTA and neomycin were administered individually.

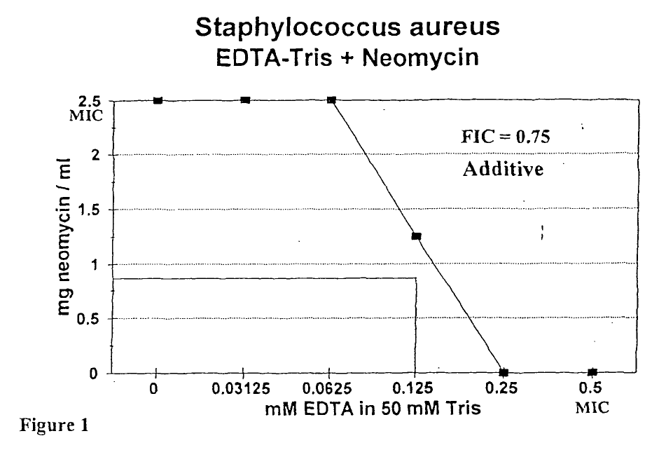

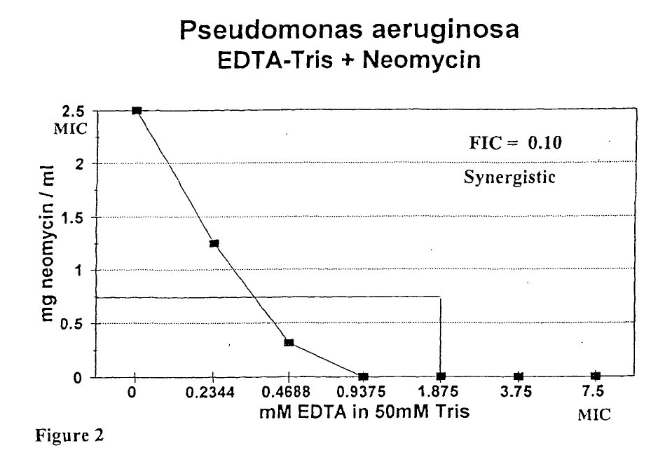

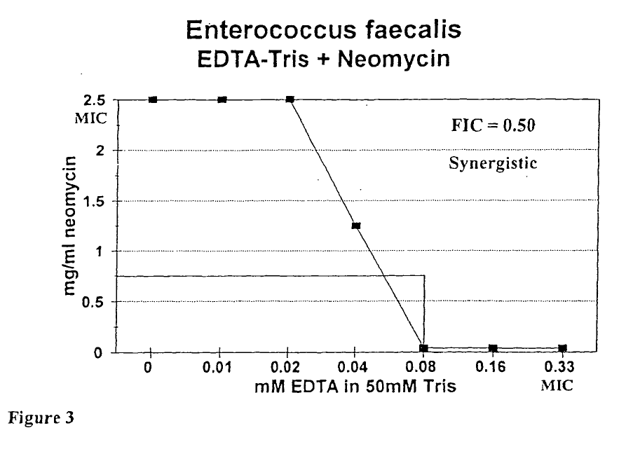

Synergistic effects were observed when various concentrations of EDTA-Tris and neomycin were reacted with Ps. aeruginosa and Ent. faecalis, while an additive effect was observed with Staph. aureus as shown in FIGS. 1-3.

### EXAMPLE 5

### Inhibition of Growth of Ps. aeruginosa and Staph. aureus

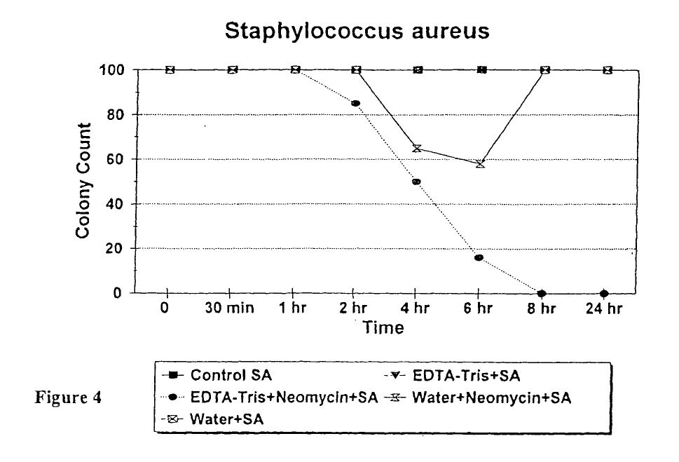

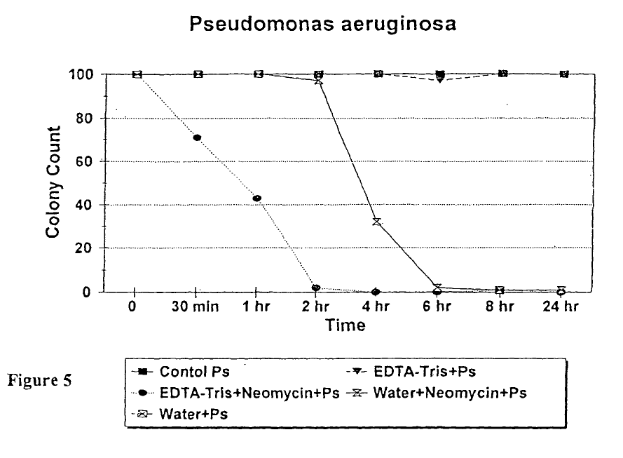

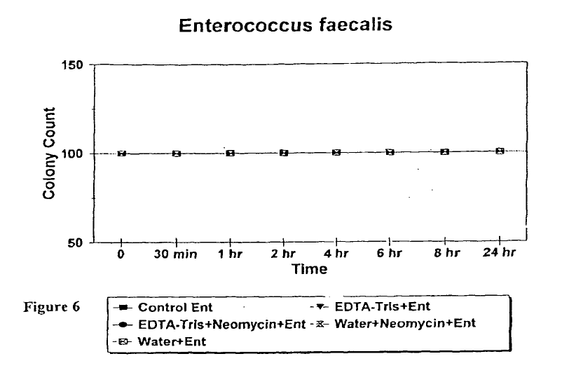

In the in vitro gauze-point-contactor study, the potentation effect was seen for EDTA-Tris-neomycin reactions with Ps. aeruginosa and Staph. aureus. These reactions are illustrated in FIGS. 4 and 5. When the same combinations of EDTA-Tris and neomycin were reacted with Ent. faecalis, no antibacterial activity was noted at these concentrations as shown in FIG. 6.

### EXAMPLE 6

### Treatment of a Skin Burn of a Dog and Antimicrobial Protection of Graft Donor Sites by Vitamin E with EDTA-Tris and Antibiotic

A mixed-breed, 35 lb spayed female canine, 1-2 years old, had been doused with gasoline, set on fire and burned over 30% of body. The dog was given initial emergency treatment for 5 days and the burned area cultured for microbial infection, identifying: β-hemolytic E. coli, Klebsiella oxytoca, Proteus sp., and Enterococcus sp. The dog was administered cefazolin systemically and the burned area cleared of tissue debris and wetted with a solution of EDTA-Tris and neomycin daily. The bum area was free of the four bacteria after 3 days of systemic and topical EDTA-Tris-neomycin therapy. After approximately 10 days, neomycin was replaced with amikacin. The dog received an autologous skin graft approximately three weeks after the burn incident and the donor site treated with EDTA-Tris-amikacin and 100 IU of Vitamin E. The dog was discharged from veterinarian hospital care two weeks later.

### EXAMPLE 7

### Treatment of Microbially Infected Skin and Oral Lesions

A composition comprising EDTA, Tris and neomycin in KY™ gel carrier was applied to skin ulcers of a turtle, a snake and a frog. Infection was reduced until eliminated, and the treated animals fully healed of their injuries and infections.

A 13 year old domestic short hair cat had developed proliferative gingivitis. The mouth was swabbed with a cotton-tipped swab twice daily for a week with a solution containing 5 mM EDTA, 50 mM Tris, and 2 mg/ml neomycin. After the first week, the mouth and gums were swabbed twice weekly for a further month. Following clearance of the infection from the animal's mouth, there was no recurrence for at least one year. A similar human oral lesion also responded to this treatment. Likewise, mouthwashes also containing EDTA, Tris and neomycin, as above, were used to treat and heal infections stomatitis of the oral cavities of iguanas and snakes.

### EXAMPLE 8

### Vitamin E Does Not Reduce the Antibacterial Efficacy of EDTA-Tris and Antibiotics

The addition of Vitamin E to solutions of EDTA-Tris enhanced the antibacterial effect of the solution. Solutions of Vitamin E alone decreased the numbers of Ps. aeruginosa significantly, as shown in Table 4.

TABLE 4
Effect of Vitamin E on the Efficacy of EDTA-Tris and
Antibiotics when Tested against Pseudomonas aeruginosa.

| Groups | Log₁₀ Colony-Forming-Units/ml |
|---|---|
| PBS | 8.20 |
| EDTA-Tris | 3.30 |
| EDTA-Tris + Neomycin | No growth |
| EDTA-Tris + Amikacin | No growth |
| Vitamin E | 6.04 |
| Vit E + EDTA-Tris | No growth |
| Vit E + EDTA-Tris + Neomycin | No growth |
| Vit E + EDTA-Tris + Amikacin | No growth |
| Vit E + Neomycin | 4.40 |
| Vit E + Amikacin | No growth |

### EXAMPLE 9

### Self-inflicted Full Thickness Flap Wound of Labial Mucosa

The patient had a self-inflicted full thickness flap wound of labial mucosa. Based on previous similar wounds untreated, it was anticipated that severe swelling and pain would persist for 7 to 10 days. The wound was rinsed with tap water and immediately contacted with. 5 mM EDTA, 50 mM Tris and 1.0 mg/ml Neomycin in an emulsion containing 100 IU Vitamin E/ml The emulsion was reapplied 3 to 4 times per day. There was minimal pain or swelling associated with the wound.

### EXAMPLE 10

### Third Degree Gasoline Burn in Dog

A dog suffering fro third degree burns received an application of 5 mM EDTA, 50 mM Tris and 1.0 mg/ml Neomycin in an emulsion containing 100 IU Vitamin E/ml applied either to bandages or as a spray 3 to 4 times a day. Continuous observation of the animal indicated that the applied composition reduced pain, as evidenced by the animal's level of relaxation, which typically would otherwise show severe stress due to degree of injury and pain associated.

### EXAMPLE 11

### Animal Inflicted Bite Wound

An animal inflicted bite wound of the human knuckle resulting in a 10 mm×7 mm full thickness flap wound of the skin penetrating to the subdermal tissues. It was expected that there would be severe pain and debilitation accompanied by limited use of the affected finger for at least a week. The wound was washed immediately with tap water and coated with emulsion containing 35 mls of hydrous lanolin, 15 mls of 333 IU/ml Vitamin E, and 0.73 gms of EDTA, 0.6 gms of Tris dissolved in 2 mls of distilled water and 0.1 mg/ml of ampicillin. The wound was washed and recoated with emulsion 3 to 4 times per day. A band-aid was used to protect the wound from additional trauma. Minimal to no pain and accelerated healing was observed compared to similar but untreated wounds.

### EXAMPLE 12

### Management of a Burn Wound

A first to second degree burn wound on the inner surface of the lower arm of approximately 1 week duration was treated. While the burn was healing, it remained crusty and pruritic. Application of 35 mls of hydrous lanolin, 15 mls of 333 IU/ml Vitamin E, and 0.73 gms of EDTA, 0.6 gms of Tris dissolved in 2 mls of distilled water and 0.1 mg/ml of ampicillin resulted in cessation of pruritis within 15 minutes. Repeat application when the wound begin to itch resulted in similar cessation of itching

### EXAMPLE 13

### Incision Wound

A composition containing 35 mls of hydrous lanolin, 15 mls of 333 IU/ml Vitamin E, and 0.73 gms of EDTA, 0.6 gms of Tris dissolved in 2 mls of distilled water and 0.1 mg/ml of ampicillin was applied once to a small painful cut on a finger. After a single application, the pain was gone along with redness. The lesion healed quickly thereafter. The medication was applied to a 1 day old very painful toe lesion that typically are sore and red for several days. After 1 application, the pain was gone and the lesion rapidly healed.

### EXAMPLE 14

### Open Abcess Wound of Cat

A cat suffering from an abscess, severe necrotizing dermatitis, fascitis and superficial myositis faced either amputation or euthanasia The wound was debrided, flushed with sterile saline and 3 gms EDTA, 2.4 gms Tris and 100 mg of ampicillin dissolved in a liter of distilled water. Initially, the wound was dressed with a wet bandage soaked in above solution. The wound was coated with an emulsion of 35 mls of hydrous lanolin, 15 mls of 333 IU/ml Vitamin E, and 0.73 gms of EDTA, 0.6 gms of Tris dissolved in 2 mls of distilled water and 0.1 mg/ml of ampicillin once per day (if a bandage was applied) or 3 to 4 times a day (no bandage was applied). The cat showed no discomfort even when the wound was left open, and a sufficient granulation bed was formed to allow surgical closure in two stages.

### EXAMPLE 15

### Formulations of Wound Prewash Solutions

Percent Cocamidopropyl betaine

| | 2.5 | 5 | 10 | 15 | 20 | 25 |
|---|---|---|---|---|---|---|
| 0.5 M EDTA | 16 μl | 16 μl | 16 μl | 16 μl | 16 μl | 16 μl |
| 1.0 M Tris | 20 μl | 20 μl | 20 μl | 20 μl | 20 μl | 20 μl |
| Deionized | 939 μl | 914 μl | 864 μl | 814 μl | 764 μl | 714 μl |
| water | | | | | | |
| Cocamido- | 25 μl | 50 μl | 100 μl | 150 μl | 200 μl | 250 μl |
| propyl | | | | | | |
| betaine | | | | | | |

Total volume = 1.0 ml of prewash.
Final concentrations of EDTA and Tris are 8 mM EDTA and 20 mM Tris.

### EXAMPLE 16

### Stock Formulations Having Increased Vitamin E Compared to Formulations With Hydrous Lanolin

(a) Oleaginous portion.

| | |
|---|---|
| Vitamin E, mixed tocopherols (333-352 IU/g) | 50 gms |
| Lecithin, soya | 50 gms |
| Sorbic acid | 0.2 gm |

Lecithin and sorbic acid were dispersed in Vitamin E oil; lecithin was dissolved over a 12-24 hour period to form an oily solution to which could be added a lipid soluble antibiotic or other agent (s).

(b) Aqueous portion:

| | |
|---|---|
| Pluronic F-127 (Poloxamer 407) | 30 gms |
| Potassium sorbate | 0.2 gm |
| Purified water, cold | 100 ml |

The Pluronic F-127 and potassium sorbate were place in a volumetric flask; add sufficient purified water to make 100 ml of solution; immediately refrigerate to keep pourable, since at room temperature a gel is formed. To the aqueous part (b), water soluble antibiotic, or other agent (s) may be added to produce the desired effect. Tris-EDTA may be added to the water at a concentration such that the final formulation administered to the patient was about 8 mM EDTA and 20 mM Tris.

Depending on the desired consistency of the fomulation (emulsion), various ratios of oleaginous and aqueous parts were prepared. For example, mixing 5 ml of each produced a soft creamy emulsion, with a low coefficient of spreading containing 176 IU/ml of Vitamin E, and 0.2% sorbic acid/potassium sorbate as a mold/yeast/fungus inhibitor (potassium sorbate is the potassium salt of sorbic acid; sorbic acid is used in oleaginous solutions, the salt in aqueous solutions).

Mixing 8 ml of the oleaginous portion (a) with 2 ml of the aqueous portion (b) also produced an emulsion with a greater amount of Vitamin E (282 IU/ml). A lower concentration of Vitamin E could also be prepared by using the ratio of 8 ml aqueous part to 2 ml oleaginous part. The effective range of Vitamin E that may be delivered by this system is approximately 70-282 IU/ml.

Lecithin as the surfactant in the above discussion was 50%; the effective concentration range for lecithin in the above formulations was 20-50 wt %; it could also be formulated to the lower to a range of about 2-50 wt %.

What is claimed is:

1. A method of wound management comprising contacting a wound of a human or animal patient with an amount of a therapeutic composition effective for promoting wound healing, the composition comprising a pharmaceutically acceptable chelating agent, a pharmaceutically acceptable pH buffering agent, a pharmaceutically acceptable antimicrobial agent, Vitamin E, a pharmaceutically acceptable carrier and a surfactant.

2. The method of claim 1 further comprising the step of washing the wound with a composition comprising a chelating agent having a concentration from about 1 mM to about 250 mM, a buffering agent having a concentration of about 10 mM to about 250 mM and a detergent having a concentration from about 1 to about 30% v/v.

3. The method of claim 2, wherein the prewash comprises about 8 mM EDTA, about 20 mM Tris, and between about 2% and about 30% v/v of cocamidopropyl betaine.

4. The method of claim 1, wherein the wound management includes pain relief.

5. The method of claim 1, further comprising debriding the wound.

6. The method of claim 1, wherein the composition is absorbed onto a medical dressing.

7. The method of claim 1, the composition further comprising an anti-inflammatory agent.

8. The method of claim 1, wherein the anti-inflammatory agent is dexamethasone.

9. The method of claim 1, the composition further comprising a pharmaceutically acceptable preservative.

10. The method of claim 1, wherein the pharmaceutically acceptable preservative is sorbic acid or a salt thereof.

11. The method of claim 1, wherein the concentrations of the chelating agent and the antimicrobial agent are selected to synergistically inhibit the proliferation of a microbial population of the wound.

12. The method of claim 1, wherein the pharmaceutically acceptable chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate.

13. The method of claim 1, wherein the pharmaceutically acceptable chelating agent is ethylenediamenetetracetic acid (EDTA).

14. The method of claim 1, wherein the effective dose of the chelating agent is between about 1 mM to about 250 mM.

15 The method of claim 14, wherein the effective dose of the chelating agent is between about 1 mM to about 100 mM.

16. The method of claim 14, wherein the effective dose of the chelating agent is between about 1 mM to about 50 mM.

17. The method of claim 1, wherein the chelating agent is EDTA having a concentration of about 8 mM.

18. The method of claim 1, wherein the pharmaceutically acceptable pH buffering agent is Tris (hydroxymethyl) aminomethane (TRIZMA Base).

19. The method of claim 1, wherein the effective dose of the buffering agent is between about 5 mM and about 250 mM.

20. The method of claim 19, wherein the effective dose of the buffering agent is between about 5 mM and about 100 mM.

21. The method of claim 20, wherein the effective dose of the buffering agent is between about 10 mM and about 100 mM.

22. The method of claim 21, wherein the effective dose of the buffering agent is about 20 mM.

23. The method of claim 1, wherein the therapeutic composition has a pH in the range of about 6.5 to about 9.5.

24. The method of claim 1, wherein the therapeutic composition has a pH of about 8.

25. The method of claim 1, wherein the antimicrobial agent is an antibiotic selected from the group consisting of a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a gramicidin.

26. The method of claim 25, wherein the antimicrobial agent is further selected from the group consisting of a β-lactam, an aminoglycoside, a vancomycin, a chloramphenicol, an erythromycin, a tetracycline, gentamicin, nalidixic acid and a streptomycin.

27. The method of claim 25, wherein the antimicrobial agent is oxytetracycline.

28. The method of claim 25, wherein the antimicrobial agent is amikacin.

29. The method of claim 25, wherein the antimicrobial agent is neomycin.

30. The method of claim 1, wherein the effective dose of the antibiotic is from about 0.04 mg/ml to about 25 mg/ml

31. The method of claim 1, wherein the effective dose of the Vitamin E is between about 20 IU/ml and 500 IU/ml.

32 The method of claim 31, wherein the effective dose of the Vitamin E is between about 50 IU/ml and 500 IU/ml.

33. The method of claim 32, wherein the effective dose of the Vitamin E is between about 100 IU/ml and about 500 IU/ml.

34. The method of claim 33, wherein the effective dose of the Vitamin E is between about 325 IU/ml and 360 IU/ml.

35. The method of claim 1, wherein the pharmaceutically acceptable carrier is non-allergenic.

36. The method of claim 1, wherein the surfactant comprises lecithin having a concentration from about 2 wt % to about 50 wt %.

37. The method of claim 1, wherein the surfactant comprises a polypropylene glycol ethoxylate having a concentration from about 5 wt % to about 30 wt %.

38. The method of claim 1, wherein the polypropylene glycol ethoxylate is Pluronic F-127 (Polaxamer 407).

39. The method of claim 1, wherein the polypropylene glycol ethoxylate is Pluronic F-127 (Polaxamer 407).

40. The method of claim 1, wherein the composition further comprises lanolin.

41. The method of claim 1, wherein the wound is a lesion of the oral mucosa of a human or animal patient.

42. The method of claim 1, wherein the wound is a lesion of the eye.

43. The method of claim 1, further comprising identifying an invasive microbial population of the wound, identifying an antibiotic capable of inhibiting the proliferation of the invasive microbial population, determining the MIC and FIC values for the antibiotic and the chelating agent; and adjusting the concentration of the antibiotic and the chelating agent of the antimicrobial composition to inhibit the proliferation of the microbial population

44 A method of wound management comprising contacting a wound of a human or animal patient with an amount of a therapeutic composition effective for promoting wound healing, the composition consisting essentially of a pharmaceutically acceptable antimicrobial agent, a pharmaceutically acceptable chelating agent, a pharmaceutically acceptable pH buffering agent, Vitamin E, a pharmaceutically acceptable carrier and a surfactant, wherein the concentrations of the chelating agent and the antimicrobial agent are selected to synergistically inhibit the proliferation of a microbial population of the wound.

45. A therapeutic composition for managing a wound, the composition comprising from about 2 to about 50 wt % of a pharmaceutically acceptable chelating agent, from about 2 to about 50 wt % of a pharmaceutically acceptable buffering agent, and a surfactant.

46. The composition of claim 45, wherein the surfactant comprises a detergent.

47. The composition of claim 45, wherein the detergent is cocamidopropyl betaine and is about 3 to about 33 wt % of the therapeutic composition.

48. The composition of claim 45, wherein the surfactant comprises lecithin from about 2-50 wt %.

49. The composition of claim 45, wherein the polypropylene glycol ethoxylate is Pluronic F-127 (Polaxamer 407).

50. The composition of claim 49, wherein the polypropylene glycol ethoxylate is Pluronic F-127 (Polaxamer 407).

51. The composition of claim 45 further comprising from about 1 to about 25 wt % of an antimicrobial agent.

52. The composition of claim 45 further comprising from about 2 to about 50 wt % of Vitamin E.

53. The composition of claim 45 further comprising from about 2 to about 98 wt % of a pharmaceutically acceptable carrier.

54 The composition of claim 45, wherein the chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate.

55. The composition of claim 54, wherein the chelating agent is ethylenediamenetetracetic acid (EDTA).

56. The composition of claim 45 further comprising 1 to 20 wt % of an anti-inflammatory agent.

57. The composition of claim 56, wherein the anti-inflammatory agent is dexamethasone.

58. The composition of claim 39, wherein the amounts of the chelating agent and the antimicrobial agent are selected to synergistically inhibit the proliferation of a microbial population.

59. The composition of claim 51, wherein the antimicrobial agent is an antibiotic selected from the group consisting of a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a gramicidin.

60. The composition of claim 59, wherein the antimicrobial agent is further selected from the group consisting of a β-lactam, an aminoglycoside, a vancomycin, a chloramphenicol, an erythromycin, a tetracycline, gentamicin, nalidixic acid and a streptomycin.

61. The composition of claim 51, wherein the antimicrobial agent is oxytetracycline.

62. The composition of claim 51, wherein the antimicrobial agent is amikacin.

63. The composition of claim 51, wherein the antimicrobial agent is neomycin.

64. The composition of claim 51, wherein the antimicrobial agent has a concentration between about 1 μg/ml and about 5 mg/ml.

65. The composition of claim 45, wherein the pharmaceutically acceptable pH buffering agent is Tris (hydroxymethyl) aminomethane (TRIZMA Base).

66. The composition of claim 45, wherein the concentration of the buffering agent is between about 5 mM and about 250 mM.

67. The composition of claim 45, wherein the concentration of the buffering agent is between about 5 mM and about 100 mM.

68. The composition of claim 45, wherein the concentration of the buffering agent is between about 10 mM and about 100 mM.

69. The composition of claim 45, wherein the concentration of the buffering agent is about 50 mM.

70. The composition of claim 52, wherein the concentration of the Vitamin E is between about 20 IU/ml and 500 IU/ml.

71. The composition of claim 52, wherein the concentration of the Vitamin E is between about 50 IU/ml and 500 IU/ml.

72. The composition of claim 52, wherein the concentration of the Vitamin E is between about 100 IU/ml and about 500 IU/ml.

73. The composition of claim 52, wherein the concentration of the Vitamin E is between about 325 IU/ml and about 360 IU/ml.

74. The composition of claim 45, further comprising a preservative.

75. The composition of claim 45, wherein the pharmaceutically acceptable carrier is non-allergenic.

76. A kit for preparing a therapeutic composition for managing a wound of an animal or human patient, comprising: packaging material containing a pharmaceutically acceptable antimicrobial agent, a pharmaceutically acceptable chelating agent, a pharmaceutically acceptable buffering agent, Vitamin E and a surfactant, and instructions directing the use of the kit for preparing a therapeutic composition for managing a wound of a human or animal.

77. The kit as in claim 75, further containing a pharmaceutically acceptable carrier.

78. The kit according to claim 75, further comprising a medical dressing, and instructions directing the use of the kit for preparing and applying the antimicrobial composition to the medical dressing and delivering the medical dressing to the wound of the human or anima.
